(19) United States
(12) Patent Application Publication
(10) Pub. No.: US 2005/0214261 A1
(43) Pub. Date: Sep. 29, 2005

(54) Method for treating a skin defect

(76) Inventors: Kazutaka Soejima, Tokyo (JP); Yumi Tanabe, Tokyo (JP); Jiro Katahira, Tokyo (JP)

Correspondence Address: ROBERT H. HAMMER III, P.C., 3121 SPRINGBANK LANE, SUITE I, CHARLOTTE, NC, 28226, US

(21) Appl. No.: 11/083,199

(22) Filed: Mar. 17, 2005

(30) Foreign Application Priority Data
Mar. 26, 2004 (JP) 2004-090768

(51) Int. Cl. A61K 45/00

(52) U.S. Cl. 424/93.7

(57) ABSTRACT

A cell-containing artificial dermis is produced by a method comprising the steps of defrosting endothelial cells and fibroblasts preserved in a frozen state, impregnating a collagen sponge with PDWHF, and dispersing the endothelial cells and fibroblasts on the collage sponge to form an artificial dermis. The invention also provides a cell-containing artificial dermis comprising a collagen sponge layer impregnated with PDWHF, and endothelial cells and fibroblasts dispersed on the surface of the collagen sponge layer, wherein the endothelial cells and fibroblasts have been preserved in a frozen state and defrosted.

### BACKGROUND OF THE INVENTION AND RELATED ART STATEMENT

1. Field of the Invention

The present invention relates to an artificial dermis for grafting and a method of producing the same. Specifically, the present invention relates to an artificial dermis and a method of producing the same containing endothelial cells, fibroblasts and growth factor.

2. Description of the Related Art

Using an artificial skin reported by Yannas, Burke et al. in 1980 given as a prototype, a two-layer artificial dermis (Integra®, USA) has been developed and clinically applied worldwide. In Japan, two kinds of artificial dermis (Pelnac®, Terudermis®) developed by improving the artificial skin of Yannas et al. have been clinically applied (for example, Japanese patent provisional publication No. 2000-262610). Deep skin defects generated by burns and trauma have been treated by using such artificial dermis in combination with grafting of autologous split thickness skin, and a large number of reports on its effectiveness have been made.

When an artificial dermis is grafted on a full-thickness skin defect where dermis is lost by burns, trauma and bedsores, autologous fibroblasts and capillaries infiltrate into it from the graft bed, to construct a dermis-like tissue different from simple granulation tissue. After the dermis-like tissue is constructed, split thickness skin is grafted on the dermis-like tissue for epidermal coverage. The skin reconstructed in this manner has improved texture with respect to elasticity and flexibility as compared with skin reconstructed with split thickness skin only. The sacrifice of skin texture and a region from which the skin was collected depends on the thickness of dermis. The significance of the existence of artificial dermis capable of artificially reconstructing dermis is that skin excellent in texture can be reconstructed with the minimum sacrifice of the region from which the skin was collected.

For successful engraftment of the skin graft, newly generated capillary vessels should infiltrate into the skin graft from the graft bed in the initial process of plasmatic imbibition in nourishment by plasma, to initiate blood circulation. For successful engraftment of split thickness skin grafted on the artificial dermis, newly generated capillary vessels from the graft bed should infiltrate via the artificial dermis into the skin graft, and when split thickness skin is grafted prior to construction of dermis-like tissue, the split thickness skin cannot be supplied with nutrients, and thus the split thickness skin is not taken. Accordingly, split thickness skin is grafted generally 2 to 3 weeks after grafting of artificial dermis in order to achieve stable engraftment.

When artificial dermis and split thickness skin are to be separately grafted, patients have to wait for a period to allow fibroblasts and capillary vessels to infiltrate into the artificial dermis to construct dermis-like tissue. However, in infants or in patients with extensive severe burns, it is difficult in some cases to prevent wounds from being infected with microorganisms during this period. If the grafted artificial dermis is infected with microorganisms, no improvement as a result of the effects of the grafted artificial dermis can be expected to occur. Further, additional skin grafting is necessary after grafting of the artificial dermis, thus increasing the mental, physical and economic burden on the patient.

Previously, there have been reports of artificial dermis consisting of collagen being made thinner or artificial dermis being provided with a large number of small holes in order to carry out simultaneous grafting of the artificial dermis and split thickness skin, but such uses deviate from the original object of artificial dermis. Under these circumstances, therefore, patients have to wait for 2 to 3 weeks for secondary skin grafting in order to attain stable skin engraftment.

Japanese patent provisional publication No. 10-80438 discloses artificial dermis comprising a silicone layer laminated on one side of a collagen sponge layer having a hollow or hole for holding skin tissues containing epidermis and dermis tissues, as well as tissue-containing artificial dermis comprising skin tissues containing epidermis and dermis tissues in the hollow or hole in the order of epidermis tissue and dermis tissue from the side of the silicone layer. Because this tissue-containing artificial dermis contains epidermis tissue etc., there is an advantage that secondary grafting of split thickness skin is not necessary. However, the epidermis tissue is collected from the healthy skin of the patient himself, which is a burden on the patient, thus resulting in the failure to spread this artificial dermis.

The present inventors revealed that when aorta-derived cultured endothelial cells, dermis-derived fibroblasts and a platelet-derived cell growth factor, that is, PDWHF (Platelet-Derived Wound Healing Factor), are simultaneously used in grafting artificial dermis onto a rat as an experimental model, infiltration of blood vessels into the artificial dermis occurs 5 days after grafting, and they further confirmed graft survival in simultaneous grafting of split thickness skin onto the artificial dermis in the same model (Journal of Japan Society of Plastic and Reconstruction Surgery, Vol. 23, No. 5, p. 291-299).

In this rat model, allogenic cultured rat cells were used. Autologous cells, on the other hand, are not practical in clinical application to trauma, particularly severe burns, because culture of the cells takes time. Accordingly, it is necessary that the same effect can be achieved by allogenic cells. Further, aorta-derived cultured endothelial cells were used in the rat model, but collection of such cells is difficult clinically, thus making it necessary to use skin-derived microvessels.

### SUMMARY OF THE INVENTION

An object of the present invention is to provide a method of producing an artificial dermis capable of using allogenic cultured dermal microvascular endothelial cells and cultured dermal fibroblasts preserved in a frozen state and capable of simultaneous grafting of artificial dermis and split thickness skin. Another object of the present invention is to provide an artificial dermis capable of using allogenic cells and capable of simultaneous grafting of artificial dermis and split thickness skin.

### DETAILED DESCRIPTION OF THE PREFERRED EMBODIMENTS

The present inventors found that when frozen and defrosted endothelial cells and fibroblasts, and PDWHF, are simultaneously used in grafting to skin defects, the infiltration of capillary vessels into artificial dermis is accelerated, and even if split thickness skin and artificial dermis are simultaneously grafted, excellent engraftment can be achieved without necrosis of the skin graft.

That is, the present invention provides a method of producing a cell-containing artificial dermis, comprising the steps of defrosting endothelial cells and fibroblasts preserved in a frozen state, impregnating a collagen sponge with PDWHF, and dispersing the endothelial cells and fibroblasts on the collage sponge to form an artificial dermis. In this method, the step of defrosting the cells preserved in a frozen state and the step of impregnating a collagen sponge with PDWHF can be conducted in this order or in reverse order. That is, the frozen endothelial cells and fibroblasts may be defrosted followed by impregnating a collagen sponge with PDWHF.

In this method, the endothelial cells and fibroblasts are desirably allogenic cells, that is, cells collected from an unrelated person.

Further, the present invention provides a cell-containing artificial dermis produced by the method.

The present invention also provides a cell-containing artificial dermis comprising a collagen sponge layer impregnated with PDWHF, and endothelial cells and fibroblasts dispersed on the surface of the collagen sponge layer, wherein the endothelial cells and fibroblasts have been preserved in a frozen state and defrosted.

The cell-containing artificial dermis of the present invention can be used to treat full-thickness skin defects with dermis hardly regenerated, not by conducting the grafting operation twice as carried out in the prior art, but by conducting the grafting operation with artificial dermis and split thickness skin only once thus reducing burden on patients, and can reconstruct skin excellent in texture with the minimum sacrifice of a region from which the skin was collected. The artificial dermis of the invention is useful particularly for operation in infants or persons of advanced age wherein a secondary operation placing mental and physical burdens is not desired, or for patients with extensive severe burns or extensive skin defects for whom it would be difficult to wait for a period for constructing dermis-like tissue after grafting of artificial dermis.

Because allogenic skin is used as a source of the endothelial cells and fibroblasts used in grafting of the artificial dermis of the invention, attention should be paid from an ethical viewpoint, but these endothelial cells and fibroblasts can be cultured in a large amount and preserved in a frozen state. In the method of the invention, PDWHF can be prepared by collecting blood from the patient himself just before operation, and when required, a necessary amount of artificial dermis is impregnated with PDWHF, and the cells are defrosted and dispersed thereon, and thus the method of the invention is an easy and practical method which can be applied widely to treatment of burns in ordinary facilities.

In this specification, the cell-containing artificial dermis refers to a dermis which contains endothelial cells and fibroblasts on the surface of a collagen sponge. The endothelial cells and fibroblasts have been subjected to freezing and defrosting treatment prior to dispersion onto the surface of a collagen sponge.

The endothelial cells are cells in the form of one layer with which the lumen of a blood vessel is covered, and the endothelial cells used in the present invention include cells derived from skin microvessels. The dermal microvascular endothelial cells are used preferably in consideration of regeneration of physiological conditions for dermis.

The fibroblasts are cells supplying fibrous components of supporting tissues and constituting an important component in fibrous connective tissues. The fibroblasts used in the present invention are desirably dermal fibroblasts, and fibroblasts derived from any site can be used.

The endothelial cells and dermal fibroblasts used are those confirmed to be free from viral infection, and cells showing normal growth etc. are used. For prevention of infection, the cells may be treated with an antibacterial agent.

The dermal microvascular endothelial cells and dermal fibroblasts are separately cultured, and upon defrosting, they are mixed and dispersed on a collagen sponge.

The endothelial cells and dermal fibroblasts are suspended in a buffer solution such as PBS and then dispersed uniformly on the surface of a collagen sponge impregnated previously with PDWHF. For example, the endothelial cells and fibroblasts are dispersed at a density of about 1×10⁵ cells respectively on a 5×5 cm collagen sponge. However, the number of endothelial cells or fibroblasts dispersed on the collagen sponge can be changed suitably depending on the age of a patient, the severeness and site of defects, etc. Usually, the cells are dispersed on only one side of the collagen sponge and grafted so as to contact this side with a graft bed.

When cells collected from the patient himself are used in grafting, the cells are called autologous cells, while when cells collected from an unrelated person, the cells are called allogenic cells.

When skin grafting is clinically conducted, culture of autologous cells is time-consuming and the autologous cells cannot be easily used in emergency cases such as trauma etc., thus making their use limited. Accordingly, the present inventors confirmed that when the endothelial cells and dermal fibroblasts after freezing and defrosting treatment are used, grafting of these cells is feasible without causing rejection even if the cells are derived from an unrelated person. A speculative reason for why rejection does not occur is that upon freezing and defrosting the cells, a structure on the cell membrane is partially destroyed to reduce antigenicity. Therefore, a special treatment against rejection is not necessary even if the cells derived from an unrelated person are used. The endothelial cells and fibroblasts can be preserved in a frozen state and defrosted as necessary before use. Accordingly, not only autologous cells but also allogenic cells supplied from a patient's blood relative or a person unrelated to the patient can be used, thus solving problems in the prior art, such as shortage of the cells and failure to supply the cells in emergency cases.

The endothelial cells and dermal fibroblasts can be frozen once before grafting and defrosted before use, but when autologous cells free of problems such as rejection etc. are used, the cells subcultured for several generations after collection can be used without subjection to freezing and defrosting treatment.

The endothelial cells and fibroblasts are divided in a suitable amount, placed in a cryo-container, suspended in a cryogenic solution, and stored usually at about −150° C.

PDWHF (platelet-derived wound healing factor) is a platelet-derived growth factor released from platelets gathering around a wound at an initial stage of wounding, to play an important role in wound healing mechanism. For preventing infection etc., PDWHF is prepared desirably from the blood of the patient who himself will receive grafting. PDWHF can be prepared usually by adding an anticoagulant such as an ACD (anticoagulant citrate dextrose) solution to the collected blood, centrifuging the blood to give platelets, and activating the platelets to release PDWHF. Other growth factors than PDWHF can be added if necessary.

The collagen sponge is obtained by extracting collagen as a major component of dermis from an animal such as cattle and synthesizing it in a sponge state. The collagen contained in the collagen sponge is desirably treated to lower antigenicity. The collagen with reduced antigenicity includes, for example, atero-collagen.

The collagen sponge used may be a commercial product for grafting. For example, Plelnac, Terudermis, Integra etc. can be used. The size and thickness of the collagen sponge can be suitably changed depending the size and site of full-thickness skin defect.

The cell-containing artificial dermis of the present invention can be applied widely to treatment of burn injury (third degree burn), injury (trauma) and scar after surgery. The artificial dermis is useful particularly in reconstruction of skin at favorite sites of scar contracture, such as neck and large joint and at sites important in orthopedic/functional reconstruction, such as exposed sites of face and hand, in cases such as extensive severe burns etc. Cases where the cell-containing artificial dermis of the present invention should not be applied include cases of wounds with infections. Accordingly, the artificial dermis is applied preferably in first resection operation at an early stage because infection of wounds always occurs in extensive severe burns. The artificial dermis of the present invention is also effective in forming granulation tissue not only by simple coverage of wounds but also by secreting or supplying the growth factor etc. contained in the artificial dermis and necessary for treatment of wounds.

From the point of view, application of the cell-containing artificial dermis of the present invention to difficult wounds such as diabetic ulcerations or venous ulcers may be beneficial.

The artificial dermis of the present invention can be grafted simultaneously with split thickness skin onto a wound. Specifically, the artificial dermis in the present application is grafted such that the surface having the cells dispersed thereon is contacted with a wound, and split thickness skin is grafted thereon. In the prior art, artificial dermis and split thickness skin are grafted separately, that is, the operation should be conducted twice, but given the cell-containing artificial dermis of the invention, a wound can be treated with the artificial dermis and split thickness skin by performing the operation only once, thus reducing the burden on patients. The artificial dermis of the invention is useful particularly for infants or persons of advanced age in reconstruction from extensive skin defects where a secondary operation placing mental and physical burdens is not desired.

However, in cases of diabetic ulcerations or venous ulcers, simultaneous grafting of skin and artificial dermis may be difficult because of wound infection and poor microcirculation of graft beds.

The split thickness skin grafted onto the artificial dermis of the invention is collected desirably from the normal skin of a patient himself because split thickness skin derived from an unrelated person is locally destroyed and not taken due to rejection. In place of split thickness skin, a sheet-shaped dermis obtained by culturing a dermis from a skin bank, a patient himself or an unrelated person can be used. However, dermis deficient in basement membrane renders graft survival difficult, thus later requiring grafting of split thickness skin from a patient himself. However, the present invention does not exclude application of the artificial dermis of the present invention together with cultured dermis.

Together with the artificial dermis, split thickness skin can be grafted as it is, but when the collected skin is smaller than an intended wound such as extensive burn, it is preferable that the split thickness skin is spread by cutting it in a mesh form before grafting and grafted in the form of a mesh.

### EXAMPLES

Hereinafter, the present invention is described in more detail by reference to the Examples, which however are not intended to limit the scope of the present invention.

### Example 1

Subject

The subject was a 63-year-old man. Due to arc discharge in electric work, his work clothes caught fire, and he received full thickness burn (dermal burn) over 49% of the body surface. Immediately, treatment for burn shock was conducted, and 20 hours after he had received the burn, resection of the burned lesion at an early stage was conducted. According to the method of the invention, the artificial dermis and split thickness skin were grafted onto 1% of the wound on the left lower leg. Because the patient was unconscious, the operation was conducted with the consent of his family.

1. Preparation of Cultured Cells

An excess of normal skin in Department of plastic surgery in Tokyo Woman's Medical University was cultured with patient's consent. The skin donor was confirmed to be negative in examination of viral infections (HIV, HCV, HBV). As bovine serum, serum proven to be negative in prion infection by the manufacturer (Gibco, USA) was used.

1) Dermal Microvascular Endothelial Cells

The skin was made free of microorganisms by treatment with antibiotics and then treated with Dispase to release epidermis, whereby dermis was obtained. The obtained dermis was cut into small sections of 2 to 3 mm in size and cultured in a dermal microvascular endothelial cell selective medium (MCDB 131 medium Gibco, USA) containing 10% fetal bovine serum (FBS) at 37° C. in 5% CO₂. After subculture for several generations, the confluent cells were recovered with trypsin from the culture dish and then suspended in a cryogenic solution (RPMI medium 1640+10% FBS+10% glycerol), pipetted into cryogenic vials in an amount of 1.0×10⁵ cells/vial and preserved in a frozen state at −150° C.

2) Dermal Fibroblasts

Dermal sections were obtained in the same manner as described above and cultured in DME medium low glucose (Gibco, USA)+10% calf serum (CS) at 37° C. in 5% CO₂. After subculture for several generations, the confluent cells were divided in an amount of 1.0×10⁵ cells/vial and preserved in a frozen state in the same manner as described above.

2. Preparation of PDWHF

From the patient, blood was collected before operation in a volume of 25 ml per section (10×10 cm) of artificial dermis used, and 2.5 ml ACD (anticoagulant citrate dextrose) solution was added to 25 ml blood. Then, erythrocytes and leucocytes were separated by centrifugation under the conditions of 1000 rpm, 20 minutes and 4° C., whereby platelet-rich plasma (PRP) was obtained. After the number of platelets therein was measured, the PRP was centrifuged again at 2100 rpm for 20 minutes at 4° C., to give platelet precipitates. The resulting platelets were washed once with HEPES, and then suspended again in HEPES at a density of 1×10⁹ platelets/ml, followed by adding thrombin (Sankyo Co., Ltd.) (1 U/ml) to activate the platelets, thus releasing PDWHF. Finally, the platelets which had released PDWHF were centrifuged under the conditions of 2400 rpm, 10 minutes and 4° C. to give a supernatant as PDWHF.

3. Preparation of Artificial Dermis

Artificial dermis (Terudermis®, collagen sponge single type, thickness 3 mm, size 10×10 cm, Terumo) was impregnated with the previously prepared PDWHF. Then, the cultured cells previously preserved in a frozen state were defrosted in such an amount that the endothelial cells and fibroblasts, each in 1 cryogenic vial (1.0×10⁵ cells), were defrosted for each 5×5 cm artificial dermis. After defrosting, the cells were washed with PBS, and then the cells in 1 cryogenic vial were re-suspended in 0.5 ml PBS. After the artificial dermis was impregnated sufficiently with PDWHF, the cell suspension was dispersed uniformly on one side of the artificial dermis.

4. Grafting Method

The prepared artificial dermis with the cell-dispersed surface down (graft surface) was grafted on full thickness defect and then split thickness skin of 10/1,000 inch in thickness was grafted thereon as a 1:3 meshed skin graft. The surface was covered with Sofratule® and fixed by tie-overdressing with Vaseline ointment.

Results

When the wound was opened on 10 days after the operation, the skin graft had been completely engrafted, and 14 days after the operation, formation of epithelium was progressing in the meshes of the netlike skin graft. Twenty days after the operation, the skin graft turned dark red, but 30 days after the operation, the skin graft had color similar to that of normal skin and felt very soft and well. Tissues were collected 10, 20 and 30 days respectively after the operation, and as a result of histological examination, bovine collagen as a component of artificial dermis was recognized to remain and slight infiltration with inflammatory cells was observed 20 days after the operation. Thirty days after the operation, however, bovine collagen had been absorbed to disappear, the inflammatory reaction had been reduced, and skin comparable to normal skin had been reconstructed.

The method of the present invention is a breakthrough invention enabling simultaneous grafting of thin split thickness skin and artificial dermis, which cannot be carried out in the prior art unless the patient should wait for 2 to 3 weeks after grafting of artificial dermis. The cells used, that is, endothelial cells and fibroblasts are both derived human dermis and considered physiologically desirable in regeneration of dermis. Further, non-autologous cells are used after preservation in a frozen state but do not show any evident rapid rejection, and can also be used in emergent cases such as trauma.

Preparation of PDWHF by collecting autologous blood requires about 80 times, but artificial dermis preparation techniques and grafting techniques are easy.

1. A method for treating a skin defect, comprising the steps of:
infiltrating PDWHF into a collagen sponge;
dispersing endothelial cells and fibroblasts on the collagen sponge; and
applying the collagen sponge on the skin defect.

2. The method according to claim 1, wherein the endothelial cells and fibroblast cells have been preserved in a frozen state and defrosted.

3. The method according to claim 1, wherein the endothelial cells and fibroblasts are allogenic.

4. The method according to claim 1, wherein the skin defect is full thickness skin defect.

5. The method according to claim 1, wherein the skin defect is burn injury, injury (trauma), diabetic limb ulceration, venous ulcers and scar after surgery.

6. The method according to claim 1, wherein the fibroblasts are dermal fibroblasts.

7. A method of producing an artificial dermis, comprising the steps of:
defrosting endothelial cells and fibroblasts preserved in a frozen state;
infiltrating PDWHF into a collagen sponge; and
dispersing the endothelial cells and fibroblasts on the collagen sponge to form an artificial dermis.

8. The method according to claim 7, wherein the endothelial cells and fibroblasts are allogenic.

9. The method according to claim 7, wherein the artificial dermis is full thickness skin defect.

10. The method according to claim 7, wherein the artificial dermis is for treating burn injury, injury (trauma), diabetic limb ulceration, venous ulcers and scar after surgery.

11. The method according to claim 7, wherein the fibroblasts are dermal fibroblasts.

12. An artificial dermis, wherein
a collagen sponge layer infiltrated PDWHF; and
endothelial cells and fibroblasts dispersing on a surface of the collagen sponge;
wherein the endothelial cells and fibroblast cells have been preserved in a frozen state and defrosted.

13. The artificial dermis according to claim 12, wherein the endothelial cells and fibroblasts are allogenic.

14. The artificial dermis according to claim 12, wherein the artificial dermis is full thickness skin defect.

15. The artificial dermis according to claim 12, wherein the artificial dermis is for treating burn injury, injury (trauma), diabetic limb ulceration, venous ulcers and scar after surgery.

16. The artificial dermis according to claim 12, wherein the fibroblasts are dermal fibroblasts.

17. An artificial dermis produced by method according to claim 7.
